(19) United States
(12) Patent Application Publication
(10) Pub. No.: US 2013/0297036 A1
(43) Pub. Date: Nov. 7, 2013

(54) Acetabular Cup

(71) Applicant: Simon Collins, Tetbury (GB)

(72) Inventor: Simon Collins, Tetbury (GB)

(73) Assignee: CORIN LIMITED, Cirencester, Gloucestershire (GB)

(21) Appl. No.: 13/811,291

(22) PCT Filed: Jul. 20, 2011
(86) PCT No.: PCT/GB2011/051379

§ 371 Date: Feb. 22, 2013

(30) Foreign Application Priority Data
Jul. 20, 2010 (GB) 1012158.0

(51) Int. Cl. A61F 2/34

(52) U.S. Cl. CPC A61F 2/34
USPC 623/22.24

(57) ABSTRACT

An acetabular cup comprises an outer shell preferably having a first tensile modulus, an inner liner preferably having at least a second tensile modulus which is greater than that of the said first tensile modulus, and at least one reinforcing element which extends from a polar region of the outer shell along a, preferably interior, surface of the outer shell. Preferably, a wall thickness of the outer shell at a region of intersection between a part-spherical portion and a frusto-conical portion of the outer shell is less at the part-spherical portion than at the frusto-conical portion. The reinforcing element has a longitudinal extent which improves the rigidity of the wall at at least the part-spherical portion.

The present invention relates to an acetabular cup for a surgical hip procedure.

Metal-on-metal (MoM) acetabular cups have been developed and used over the past decade and were primarily a response to unacceptable wear and debris from conventional Ultra High Molecular Polyethylene (UHMWPE) acetabular prostheses. MoM bearings however suffer from their own limitations, such as metal ion release that may cause clinical issues.

More recently there has been a general trend in orthopaedics of using increased bearing diameters for hip replacements as they can reduce wear and reduce the incidence of post-operative dislocation. In an attempt to make these as thin as possible, issues are encountered with undesirable flex, causing the outer shell to be often deformed into an oval or elliptical shape during insertion by the surgeon. This of course is unsuitable when then trying to securely engage a part-spherical inner liner via the commonly used taper arrangement found in these offerings.

It has been suggested that a one piece fully ceramic acetabular cup can be utilised for hip replacements and thus may prevent this flexing found to be an issue. However, ceramic is found to be overly hard, having a high elastic or tensile modulus, and therefore provides poor stress-shielding for the natural bone interface. This results, over time, in the bone interface potentially resorbing and the fixation of the outer shell weakening. The benefit of a ceramic component, however, is that the cup wall thickness can be reduced whilst still maintaining the necessary rigidity to withstand the impaction forces and subsequent biomechanical loads.

The present invention seeks to provide a solution to these problems.

According to a first aspect of the present invention, there is provided an acetabular cup comprising an outer shell, an inner liner, and at least one reinforcing element which extends from a polar region of the outer shell along a surface of the outer shell.

Preferable and/or optional features of the first aspect of the invention are set forth in claims 2 to 29, inclusive.

According to a second aspect of the invention, there is provided an inner liner for an acetabular cup, the inner liner comprising an articulating bearing inner surface and an outer surface having at least one elongate channel which extends from a polar region of the outer surface and towards or substantially towards a rim.

Preferable and/or optional features of the second aspect of the invention are set forth in claims 32 to 43, inclusive.

The present invention will now be more particularly described, by way of example only, with reference to the accompanying drawings, in which:

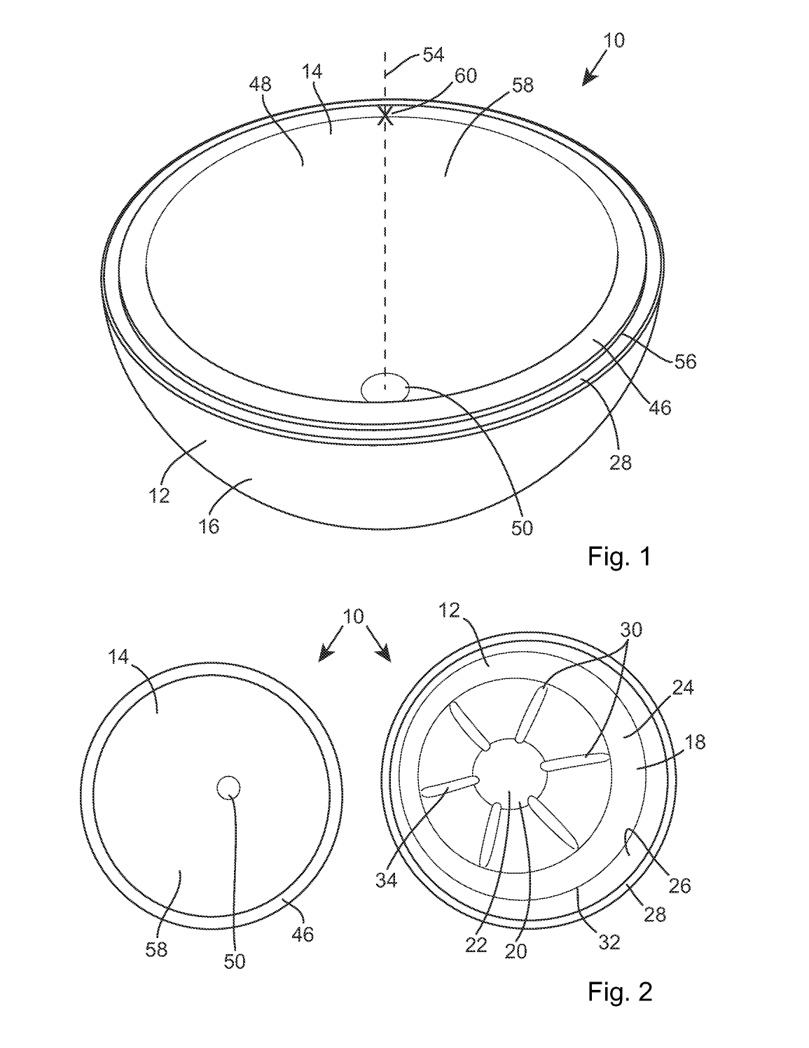

FIG. 1 shows a perspective view of one embodiment of an acetabular cup, in accordance with the present invention;

FIG. 2 is a top plan view showing the acetabular cup of FIG. 1, with an outer shell and an inner liner separated;

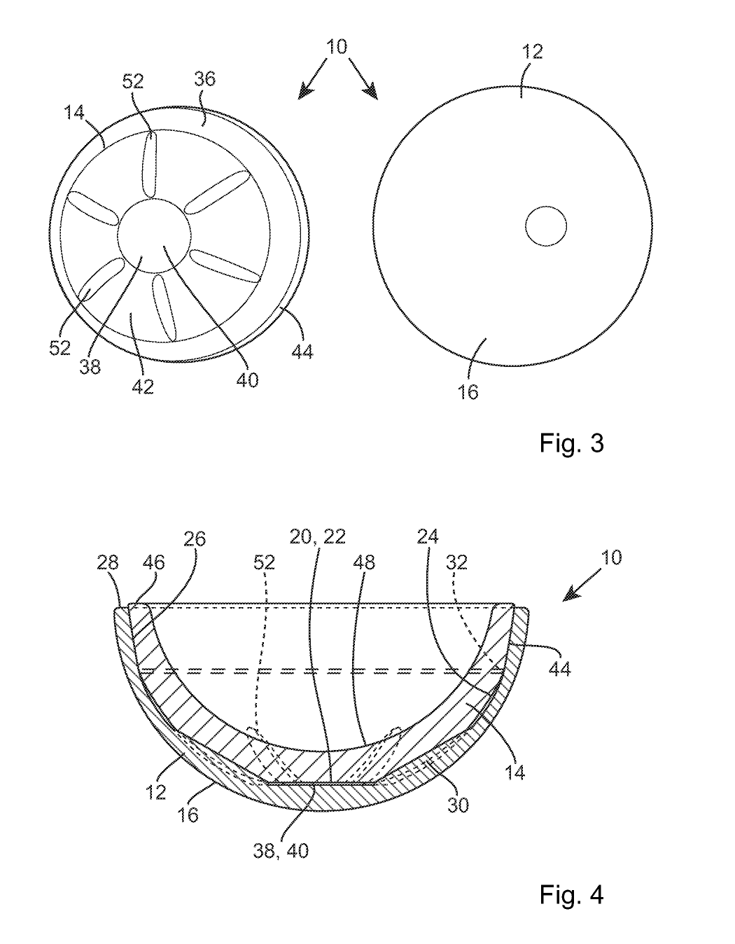

FIG. 3 is a bottom plan view of the separated outer shell and the inner liner; and

FIG. 4 is a cross-sectional view along the polar plane of the assembled acetabular cup, shown in FIG. 1.

Referring to the drawings, there is shown one embodiments of a two-piece acetabular cup 10 which comprises an outer shell 12 dimensioned to fit a prepared natural acetabulum of a patient, and an inner liner 14. The outer shell 12 is formed of a first material having a first tensile modulus. Preferably, the outer shell 12 is formed of bio-compatible metal, such as titanium or an alloy thereof such as Ti₆Al₄V which has a beneficial combination of strength, corrosion resistance, weld and fabricability. Also preferably, the outer shell 12 has a density of approximately 4500 kg/m3, tensile modulus of 110 GPa, and tensile strength of 1000 MPa. However, plastics such as UHMWPE, or ceramics can be considered.

A bone-interfacing outer surface 16 of the outer shell 12 is preferably part-spherical or substantially part-spherical. One or more splines, ribs, tabs, lugs or fixation elements may be integrally formed as one-piece on the outer surface 16 to aid engagement with the prepared natural bone of the patient's acetabulum.

An interior surface 18 of the outer shell 12 has a polar region 20 which may include a flat 22, a part-spherical surface portion 24 which extends from the polar region 20, and a frusto-conical surface portion 26 which extends contiguously from the part-spherical surface portion 24 to a rim 28 of the outer shell 12. It is feasible that at least one of the flat 22 of the polar region 20, the part-spherical surface portion 24 and the frusto-conical surface portion 26 may be dispensed with in favour of one or more of the other.

The wall thickness at the flat 22 of the polar region 20 typically increases in thickness towards the polar axis, due to the part-spherical outer surface 16.

To optimise the dimension of the acetabular cup 10, preferably a wall thickness of the outer shell 12 is in the range of approximately 1 mm to 1.5 mm. This may be an average wall thickness, or may be a constant. More preferably, this wall thickness is at or adjacent to the rim 28. This provides a very thin outer shell 12, which thus may lead to undesirable flex. As such, a plurality of spaced reinforcing elements 30 is provided on the outer shell 12. Each reinforcing element 30 is, in this embodiment, integrally formed as one-piece with the interior surface 18 of the outer shell 12. This is preferable, since it also allows support and engagement for the inner liner, as discussed herein. However, the reinforcing elements could be, additionally or alternatively, on the outer surface 16 of the outer shell 12.

Each reinforcing element 30 extends from the polar region 20 of the outer shell 12 and along the interior surface 18. In this embodiment, each reinforcing element 30 has a rectilinear longitudinal extent, but other shapes such as curved, serpentine, or spiral can be considered as necessity dictates. If also on the outer surface 16, the positioning may be mirrored relative to the interior reinforcing elements or angularly offset relative thererto.

Each reinforcing element 30 extends radially outwards from the polar region 20. In this case, each reinforcing element 30 starts from the flat 22 of the polar region 20 and ramps away therefrom onto the part-spherical surface portion 24. The reinforcing element 30 terminates partway along the part-spherical surface portion 24, typically beyond the midway circumferential line and prior to the frusto-conical surface portion 26.

However, an area of weakness and thus potential undesirable flex occurs at the intersection between the part-spherical surface portion 24 and the frusto-conical surface portion 26. This is due to the curve of the part-spherical outer surface 16 of the outer shell 12, and the flat interior frusto-conical surface portion 26 intersecting with the curved interior part-spherical surface portion 24. This configuration results in the wall thickness of the outer shell 12 at the frusto-conical surface portion 26 decreasing towards the intersection with the part-spherical surface portion 24. This potential area of weakness at the region of intersection 32, and/or the lower rigidity of the part-spherical surface portion 24 relative to the frusto-conical surface portion 26 may thus be offset by the use of the reinforcing elements 30. It may therefore be preferable that the reinforcing elements 30 extend to or adjacent to the region of intersection 32, or even to or adjacent to the frusto-conical surface portion 26. The reinforcing elements 30 may even extend onto the frusto-conical surface portion 26 and terminate thereon. In the event that the flat and/or the frusto-conical surface portions are dispensed with, the reinforcing elements extend from the polar region and may extend to or adjacent to the rim.

Termination of the reinforcing elements 30 is preferably tapered or ramped. However, termination may be at a defined shoulder to aid positive engagement.

The reinforcing elements 30 preferably have a flat or planer upper surface, ridgeline, apex or crest 34 along their longitudinal extents. The reinforcing elements 30 also preferably have curved lateral extents along their longitudinal extents. Consequently, each reinforcing element 30 has a non-uniform lateral cross-sectional area along its longitudinal extent to accommodate at least the curvature of the part-spherical surface portion 24.

In this embodiment, the reinforcing elements 30 are equi-angularly spaced apart from each other. However, non-equi-angular spacing can be considered, for example to enable intra-operative selection from a plurality of different possible said outer shells 12 on a patient by patient basis. This would be beneficial where extra rigidity of the outer shell 12 and/or tailored stress-shielding may be advantageous at certain points or regions of the outer shell 12 to supplement a particular patient's bone structure, density or condition.

The inner liner 14, in this embodiment, is formed of a second material having at least a second tensile modulus which is greater than that of the first tensile modulus of the said first material of the outer shell 12. Preferably, the inner liner 14 is formed of a hard bearing bio-compatible material, such as ceramic. More preferably, the inner liner 14 is entirely formed as one-piece of the said hard bearing bio-compatible material. However, it is possible that the inner liner 14 can be formed from bio-compatible metal as above, with a hard bearing articulating surface such as a ceramic layer thereon. This latter option, in the present invention, may not be most preferable, since a unitary ceramic inner liner 14 allows for a decreased wall thickness and thus an overall reduction in the dimensions of the acetabular cup 10, which is important. Other options for the inner liner include plastics such as UHMWPE, and metal.

The second tensile modulus is preferably or substantially 370 GPa.

An outer-shell interfacing outer surface 36 of the inner liner 14 is complementarily shaped to be receivable in the outer shell 12 so that the inner liner 14 is seatable thereon to either provide for tapered engagement, push-fit engagement, a tolerance fit, or bonded engagement. To this end, the outer surface 36 has a polar region 38 which may be curved and/or include a flat 40, a part-spherical surface portion 42 which extends from the polar region 38, and a frusto-conical surface portion 44 which extends contiguously from the part-spherical surface portion 42 to a rim 46 of the inner liner 14.

The interior bearing articulating surface 48 of the inner liner 14 is preferably entirely part-spherical from and including the interior polar region 50 to the rim 46, and is dimensioned to receive the ball of a femoral head. The articulating surface 48 may be adapted to accept the ball of the femoral head either in a captive or non-captive manner, dependent on necessity. Again, by providing a range of different inner liners 14 having a common interface with the or each said outer shell 12, selection and in particular intra-operative selection is facilitated based on a particular patient's requirements.

The outer surface 36 of the inner liner 14, in this embodiment, includes a plurality of spaced recessed channels 52 complementarily shaped to receive at least part of a respective reinforcing element 30. Each recessed channel 52 extends radially from the polar region 38 of the outer surface 36 of the inner liner 14. Preferably, each channel 52 extends from the flat 40 of the polar region 38 and partway along the part-spherical outer surface 36. However, as with the outer shell 12, each channel 52 may extend to or adjacent to the frusto-conical surface portion 44 of the inner liner 14.

The recessed channels 52 are angularly spaced to match the spacing of the reinforcing elements 30, and a depth is sufficient so that preferably only part of the lateral extent of each reinforcing element 30 in the polar plane having the polar axis 54 is receivable therein. In other words, a lateral cross-sectional area of each channel 52 along its longitudinal extent is less than the corresponding lateral cross-sectional area of the reinforcing element 30. The reinforcing elements 30 thus, in this case, extend laterally and longitudinally from the channels 52 when received therein.

Due to the depth of the channels 52, the inner liner 14 is supported by the reinforcing elements 30 so that the polar region 38 and at least part of the part-spherical surface portion 42 spaced from the frusto-conical surface portion 44 of the outer surface 36 of the inner liner 14 are spaced from the polar region 20 and corresponding part of the part-spherical surface portion 24 of the interior surface 18 of the outer shell 12.

As with the outer shell 12, it is feasible that at least one of the flat 40 of the polar region 38 of the outer surface 36 of the inner liner 14, the part-spherical surface portion 42 and the frusto-conical surface portion 44 may be dispensed with in favour of one or more of the other.

The recessed channels 52 in the outer surface 36 of the inner liner 14 are preferable, since it facilitates indexing and/or positive rotational engagement of the inner liner 14 relative to the outer shell 12. However, the recessed channels 52 may be omitted. In this case, the reinforcing elements 30 may simply support the inner liner 14 via point or portion contact along their longitudinal extents.

Since engagement primarily occurs between the outer shell 12 and inner liner 14 at the respective frusto-conical surface portions 44, 26, if the outer shell 12 is sufficiently strong, then the inner liner 14 may be supported so as to be entirely spaced from the reinforcing elements 30.

The inner liner 14 preferably has a minimum wall thickness of or substantially 3 mm.

In order to optimise the dimensions of the acetabular cup 10 so that the rim 56 of the cup 10 is minimised whilst maintaining or maximising an opening 58 of the inner liner 14 for receiving a ball of the femoral head, the rim 46 of the inner liner 14 preferably terminates in the polar plane having the polar axis 54 at 165 degrees from the equatorial centre 60. Due to the outer surface 36 of the inner liner 14 having the frusto-conical surface portion 44 extending from the rim 46, and having the part-spherical interior articulating surface 48, by providing the rim 46 so as to be spaced from the equatorial line, i.e. so that the interior articulating surface 48 is not hemispherical or a full hemisphere, and thereby slightly reducing the depth of the inner liner 14, a radial thickness of the rim 46 is reduced and the opening 58 to the interior articulating surface 48 of the inner liner 14 is effectively increased.

The termination of the articulating surface 48 at 165 degrees form the equatorial centre 60 substantially equates to offsetting the plane of the rim 46, being normal to the polar axis 54, towards the interior polar region 50 of the inner liner 14 by or substantially by 3 mm along the polar axis 54 from the equatorial centre 60.

Although 165 degrees is preferred, the offset may be in the range of 150 degrees to 175 degrees, and this may be due at least in part to the different sizes of cup used selected based on the patient. Additionally, or alternatively, although an offset of 3 mm is preferred, the offset may be in the range of 1.5 mm to 5 mm.

The polar regions mentioned above include the respective polar axes. Each polar region extends preferably radially and preferably uniformly from its respective polar axis to provide a diameter of up to or substantially 25 mm. The diameter may, however, be less. If the polar region includes the optional flat, whether this is on the outer surface and/or the inner surface, a diameter of the flat is preferably up to or substantially 20 mm, and is preferably uniform about the included polar axis. The flat may, however, be less. The flat may also overlap its included polar region and/or the polar region of another surface of the acetabular cup.

In a modification to the above embodiments, the inner liner may include one or more of the elongate channels and/or additional recessed channels in order to reduce weight. This may be exclusive of the reinforcing elements of the outer shell. In this way, such an inner liner can be used with other kinds of outer shell and/or intermediate liner which may be devoid of the reinforcing elements or have the reinforcing elements on a different surface which is spaced away or remote from the surface comprising the or each recessed channel. It is beneficial to the patient to conserve weight when a prosthesis is used, whilst also reducing materials and thus cost. The configuration of the or each recessed channel can be as described herein with referenced to the reinforcing elements. However, it is preferable that the recessed channel extends from the polar region of the outer surface of the inner liner and preferably at least generally in the longitudinal direction of the polar axis of the inner liner towards or generally in the direction of the rim of the inner liner.

In this modification, the outer surface of the inner liner may include other flats which are spaced from or overlaps the polar region, dependent on the outer shell to be used. The channels may be interconnected, for example, at the polar region, or may be separate of each other. The channels, whether separate or interconnected, may be equi-angularly spaced apart, or may be non-uniformly spaced apart or of a non-uniform length dependent on the patient and the requirements. In other words, more material may be required for strength purposes at certain positions around the inner liner, based on the patient, and thus this may influence the number, positioning, size and spacing of the or each channel.

As above, one or more of the flat at the polar region, part-spherical surface portion and frusto-conical surface portion may be dispensed with in favour of one or more of the remaining or a differently shaped surface portion.

The weight saving channel is applicable to ceramic liners, either being wholly ceramic or with a ceramic interior articulating surface. However, the channel can be utilised with liners of other materials, such as metal and plastics.

Although preferably a range of outer shells and inner liners having a common interface to allow interchangability is provided, the outer shell and inner liner may be provided pre-assembled and/or may be non-separable following engagement.

The reinforcing elements are preferably integrally formed as one-piece with the outer shell. However, the reinforcing elements could be provided as a separate web which may be affixed, for example, by bonding, to the interior surface of the outer shell.

Although a plurality of spaced reinforcing elements are provided on the outer surface and/or inner surface of the outer shell, a single reinforcing element may be utilised. In this case, the reinforcing element may extend from the polar region and only to one side or direction of the outer shell. Alternatively, the reinforcing element may extend from one side or direction of the outer shell, through the polar region and to another side or direction. Furthermore, the reinforcing element may by a single element with a plurality of interconnected arms which extend from a hub portion of the reinforcing element at the polar region, distal ends of the arms being spaced part.

The or each reinforcing element preferably extends in or in parallel with the polar plane of the acetabular cup and/or its own polar plane. However, one or more reinforcing elements may be longitudinally rectilinear but extending at an angle or non-parallel, for example, 15 degrees, to the polar plane so that the longitudinal axis extends through the polar plane. This arrangement may be used in combination with other configurations of the reinforcing elements, and may be beneficial in allowing additional reinforcement at certain sections or portions of the outer shell.

Similarly, the or each channel preferably extends in or in parallel with the polar plane of the acetabular cup and/or its own polar plane. However, one or more of the channels may be longitudinally rectilinear but extending at an angle or non-parallel, for example, 15 degrees, to the polar plane so that the longitudinal axis extends through the polar plane. This arrangement may be used in combination with other configurations of the channels either to complement the reinforcing elements or if used alone to aid in weight reduction.

One or more of the said channels may also be curved or include a curved portion, and again this would be beneficial in targeting specific areas of the liner for weight reduction and/or for other purposes, for example, if being used in combination with the reinforcing elements of the outer shell.

An intermediate liner which is interposed between the inner liner and the outer shell may also be utilised. In this case, the inner liner is seatable for engagement in the intermediate liner, which in turn is seatable for engagement in the outer shell.

The outer surface of the outer shell may also include a bone-fixation portion, such as a separate coating of hydroxyapetite or an integrally formed porous surface portion, such as formed by casting, sintering or laser, to encourage bone growth thereon.

Tensile modulus mentioned herein and throughout is Young's modulus and may be referred to as modulus of elasticity.

The above described acetabular cup may be utilised in one or both of a total head replacement procedure and a hip resurfacing procedure.

It is thus possible to provide an acetabular cup having a metal outer shell and a ceramic inner liner. It is also possible to reduce a wall thickness of the outer shell by the use of reinforcing elements, which prevent or limit an increase in flex. Stress-shielding as well as bone fixation is also improved by the use of the metal outer shell. Without altering the wall thickness, it is further possible to widen an opening of the inner shell by offsetting the rim of the inner shell towards the polar region and away from the equatorial centre. An acetabular cup therefore having a thin but strong outer shell as well as a smaller overall outside diameter at the rim is possible.

The embodiments described above are provided by way of examples only, and various other modifications will be apparent to persons skilled in the field without departing from the scope of the invention as defined by the appended claims.

1. An acetabular cup comprising an outer shell, an inner liner, and at least one reinforcing element which extends from a polar region of the outer shell along a surface of the outer shell.

2. The acetabular cup as claimed in claim 1, wherein the outer shell has a first tensile modulus, and the inner liner has at least a second tensile modulus which is greater than that of the said first tensile modulus.

3. The acetabular cup as claimed in claim 1, wherein the reinforcing element extends from the polar region of the outer shell along an interior surface of the outer shell.

4. The acetabular cup as claimed in claim 1, wherein a plurality of said reinforcing elements are provided, each extending from the polar region of the outer shell.

5. (canceled)

6. The acetabular cup as claimed in claim 3, wherein the interior surface of the outer shell includes a part-spherical portion which extends from the polar region, and a frusto-conical portion which extends from or substantially from a rim of the outer shell, the said at least one reinforcing element extending towards the frusto-conical portion.

7. The acetabular cup as claimed in claim 5, wherein the reinforcing element extends along the part-spherical portion to or substantially to the frusto-conical portion.

8. The acetabular cup as claimed in claim 5, wherein a wall thickness of the outer shell at the region of intersection between the part-spherical portion and the frusto-conical portion increases in a direction of the frusto-conical portion, the reinforcing elements having a longitudinal extent which improves the rigidity of the wall at at least the part-spherical portion.

9. The acetabular cup as claimed in claim 1, wherein the polar region includes a flat, and the said at least one reinforcing element extends from the flat to a part-spherical portion.

10. The acetabular cup as claimed in claim 8, wherein a wall thickness of the outer shell at the polar region increases towards the polar axis.

11. The acetabular cup as claimed in claim 1, wherein a longitudinal extent of the said at least one reinforcing element is rectilinear.

12. (canceled)

13. (canceled)

14. (canceled)

15. The acetabular cup as claimed in claim 1, wherein the said at least one reinforcing element has a non-uniform lateral cross-sectional area along its longitudinal extent.

16. (canceled)

17. The acetabular cup as claimed in claim 1, wherein the inner liner is supportable by the said at least one reinforcing element so that in use a polar region of the inner liner is held in spaced relationship with the polar region of the outer liner.

18. The acetabular cup as claimed in claim 1, wherein an outer surface of the inner liner includes a complementary channel for at least in part receiving the said at least one reinforcing element.

19. The acetabular cup as claimed in claim 13, wherein the channel is rectilinear and extends from or substantially from the polar region of the outer surface of the inner liner.

20. (canceled)

21. (canceled)

22. (canceled)

23. The acetabular cup as claimed in claim 13, wherein a lateral cross-sectional area of the channel is less than a lateral cross-sectional area of the reinforcing element, so that the in use reinforcing element sits proud of the respective channel.

24. (canceled)

25. The acetabular cup as claimed in claim 1, wherein an inner surface of the inner liner is a part-spherical articulating surface for a femoral head.

26. The acetabular cup as claimed in claim 16, wherein the interior surface of the inner liner at the rim terminates in the polar plane in a range of 150 degrees to 175 degrees from the equatorial centre, so that the inner liner has a shallower depth and due to the part-spherical interior surface a radially thinner rim thereby increasing an opening for a femoral head.

27. The acetabular cup as claimed in claim 16, wherein the interior surface of the inner liner at the rim terminates in the polar plane in a range of 1.5 mm to 5 mm along the polar axis from the equatorial centre, so that the inner liner has a shallower depth and due to the part-spherical interior surface a radially thinner rim thereby increasing an opening for a femoral head.

28. The acetabular cup as claimed in claim 1, wherein the inner liner is ceramic.

29. The acetabular cup as claimed in claim 1, wherein the outer shell is metal.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)
